(19) United States
(12) Patent Application Publication
(10) Pub. No.: US 2008/0200816 A1
(43) Pub. Date: Aug. 21, 2008

(54) ULTRASOUND DIAGNOSTIC APPARATUS, ENDOSCOPE AND ULTRASOUND TRANSDUCER

(75) Inventor: Takanao FUJIMURA, Sagamihara-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/034,304

(22) Filed: Feb. 20, 2008

(30) Foreign Application Priority Data
Feb. 21, 2007 (JP) 2007-041290

(51) Int. Cl. A61B 8/14; A61B 1/018

(52) U.S. Cl. 600/472; 600/104

(57) ABSTRACT

An ultrasound diagnostic apparatus of the present invention includes a cap, and an ultrasound transducer which includes a lens provided in a piezoelectric element, is rotatable and swingable and is provided in a cap, and the lens focuses ultrasound transmitted from the piezoelectric element and includes a shape in which a first focal length of a first lens surface on a section parallel with a rotational center axis including a center of a sound beam axis of the ultrasound is shorter than a second focal length of a second lens surface on a section orthogonal to the rotational center axis including the center of the sound beam axis.

This application claims benefit of Japanese Application No. 2007-041290 filed in Japan on Feb. 21, 2007, the contents of which are incorporated by this reference.

### BACKGROUND OF THE INVENTION

1. Field of the Invention

The present invention relates to an ultrasound diagnostic apparatus including, in a cap, an acoustic medium in which a sound velocity when ultrasound passes is lower than in a medium outside the cap, and an ultrasound transducer rotatable around an ultrasound transducer rotational axis of the cap, an endoscope and the ultrasound transducer.

2. Description of the Related Art

An ultrasound diagnostic apparatus which repeatedly transmits ultrasound to a cavity wall to be a subject part in a cavity from an ultrasound transducer, receives an echo signal of the ultrasound reflected from the cavity wall of the subject part with the ultrasound transducer, displays an ultrasound image that is a two-dimensional visible image of the subject part on a screen of a display device, and can be used for diagnosis or the like of a lesion portion is known, and various kinds of such apparatus have been proposed.

As devices used in combination with the ultrasound diagnostic apparatus, an ultrasound endoscope, an ultrasound probe and the like are known. For example, as an ultrasound endoscope including the ultrasound diagnostic apparatus, the one with a configuration including an endoscope observation unit for obtaining an endoscope image of a subject part and an ultrasound observation unit for obtaining an ultrasound image of the subject part, at a distal end portion of an insertion portion which is inserted into a cavity is known.

The ultrasound observation unit is provided at a distal end side in an insertion direction from the endoscope observation unit, for example, in the distal end portion of the insertion portion, and is configured by including a distal end cap covering the distal end portion of the insertion portion (hereinafter, simply called a cap), an ultrasound transducer provided rotatably inside the cap around an ultrasound transducer rotational axis which will be described later of the cap, and an acoustic medium filled between the ultrasound transducer and an inner surface of the cap inside the cap.

The ultrasound transducer is configured by including a piezoelectric element which is an electromechanical transducer element, electrodes which are provided on one surface of the piezoelectric element and on the other surface on a side opposite from the one surface, and apply voltage to the piezoelectric element or receive voltage from the piezoelectric element, an acoustic lens (hereinafter, simply called a lens) which focuses ultrasound transmitted with a vibration of the piezoelectric element as a sound source, a backing material which is provided on the electrode on the other surface of the piezoelectric element and attenuates unnecessary ultrasound, and a housing which holds the piezoelectric element, the electrodes, the lens and the backing material with the lens exposed, and configures a casing of the ultrasound transducer.

Here, as the above described acoustic medium, water is generally used so that the sound velocity at which ultrasound passes becomes the same as in water, body fluid and the like which are media outside the cap in the cavity existing outside the cap. However, for the purpose of enhancing smoothness of rotation of the ultrasound transducer, preventing bubbles which occur in the acoustic medium with rotation of the ultrasound transducer, and the like, a configuration of using an oil medium such as liquid paraffin as the acoustic medium is known, and the one is disclosed in, for example, Japanese Patent Laid-Open No. 7-241293.

### SUMMARY OF THE INVENTION

To be brief an ultrasound diagnostic apparatus of the present invention comprises a cap whose outer periphery contacts a cavity wall directly or via a medium outside the cap, and an ultrasound transducer including an. electromechanical transducer element and an acoustic lens provided on the electromechanical transducer element, rotatable or swingable, capable of transmitting ultrasound with the electromechanical transducer element as a sound source and/or receiving echo from a subject area, and provided in the cap, in which the lens focuses the ultrasound transmitted from the electromechanical transducer element, and includes a shape in which a first focal length of a first lens surface on a section parallel with an ultrasound transducer rotational axis or a swing axis including a center of a sound beam axis of the ultrasound is shorter than a second focal length of a second lens surface on a section orthogonal to the ultrasound transducer rotational axis or the swing axis including the center of the sound beam axis.

Further, an endoscope of the present invention comprises a cap which is provided on a distal end of an insertion tube, and has an outer periphery contacting a cavity wall directly or via a medium outside the cap, and an ultrasound transducer comprising an electromechanical transducer element, and a lens provided on the electromechanical transducer element, rotatable or swingable, capable of transmitting ultrasound with the electromechanical transducer element as a sound source and/or receiving echo from the subject area, and provided in the cap, in which the lens focuses the ultrasound transmitted from the electromechanical transducer element, and includes a shape in which a first focal length of a first lens surface on a section parallel with an ultrasound transducer rotational axis or a swing axis including a center of a sound beam axis of the ultrasound is shorter than a second focal length of a second lens surface on a section orthogonal to the ultrasound transducer rotational axis or the swing axis including the center of the sound beam axis.

Further, an ultrasound transducer of the present invention comprises an ultrasound transducer unit comprising an electromechanical transducer element, a lens provided on the electromechanical transducer element, rotatable or swingable, and capable of transmitting ultrasound with the electromechanical transducer element as a sound source and/or receiving echo from a subject area, and provided in the cap, in which the lens focuses the ultrasound transmitted from the electromechanical transducer element, and includes a shape in which a first focal length of a first lens surface on a section parallel with an ultrasound transducer rotational axis or a swing axis including a center of a sound beam axis of the ultrasound is shorter than a second focal length of a second lens surface on a section orthogonal to the ultrasound transducer rotational axis or the swing axis including the center of the sound beam axis.

The above and other objects, features and advantages of the invention will become more clearly understood from the following description referring to the accompanying drawings.

### BRIEF DESCRIPTION OF THE DRAWINGS

FIG. 1 is a view showing a configuration of an ultrasound endoscope apparatus including an ultrasound diagnostic apparatus of the present embodiment;

FIG. 2 is a perspective view schematically showing in enlarged manner a distal end portion of an insertion portion of an ultrasound endoscope of FIG. 1;

FIG. 3 is a partial cross-sectional view taken along line III-III in FIG. 2 showing an ultrasound transducer to be seen from a lens surface side;

FIG. 4 is a partial cross-sectional view showing a configuration of the ultrasound transducer in FIG. 3 with a cap from a direction differing from FIG. 3 by 90 degrees;

FIG. 5 is a perspective view showing focal lengths of a first lens surface and a second lens surface in a lens of an ultrasound transducer unit in FIG. 4;

FIG. 6 is a perspective view showing a state in which the focal lengths of the first lens surface and the second lens surface correspond to each other in a state in which the ultrasound transducer unit in FIG. 5 is provided in the cap with an acoustic medium;

FIG. 7 is a cross-sectional view taken along line VII-VII in FIG. 6;

FIG. 8 is a cross-sectional view taken along line VIII-VIII in FIG. 6;

FIG. 9 is a perspective view showing a configuration of the ultrasound transducer unit of the ultrasound transducer included by the ultrasound diagnostic apparatus showing the present embodiment;

FIG. 10 is a view showing a lens shape in FIG. 9 with a section of the first lens surface and a section of the second lens surface;

FIG. 11 is a view showing a partial cross-sectional view taken along line XIk-XIk in FIG. 2 with a partial cross-sectional view taken along line XIk-XIk in FIG. 2;

FIG. 12 is a view showing a lens shape differing from that of FIG. 10 of the ultrasound transducer unit of the ultrasound transducer included by the ultrasound diagnostic apparatus showing the present embodiment with the section of the first lens surface and the section of the second lens surface;

FIG. 13 is a view showing a lens shape differing from that of FIG. 12 with the section of the first lens surface and the section of the second lens surface;

FIG. 14 is a partial cross-sectional view showing focal positions of the first lens surface and the second lens surface in the present embodiment;

FIG. 15 is a view showing a lens shape differing from those of FIGS. 10 and 12 of the ultrasound transducer unit of the ultrasound transducer included by the ultrasound diagnostic apparatus showing the present embodiment with the section of the first lens surface and the section of the second lens surface;

FIG. 16 is a view showing a lens shape differing from those of FIGS. 10, 12 and 15 of the ultrasound transducer unit of the ultrasound transducer included by the ultrasound diagnostic apparatus showing the present embodiment with the section of the first lens surface and the section of the second lens surface;

FIG. 17 is a view showing a lens shape differing from those of FIGS. 10, 12, 15 and 16 of the ultrasound transducer unit of the ultrasound transducer included by the ultrasound diagnostic apparatus showing the present embodiment with the section of the first lens surface and the section of the second lens surface;

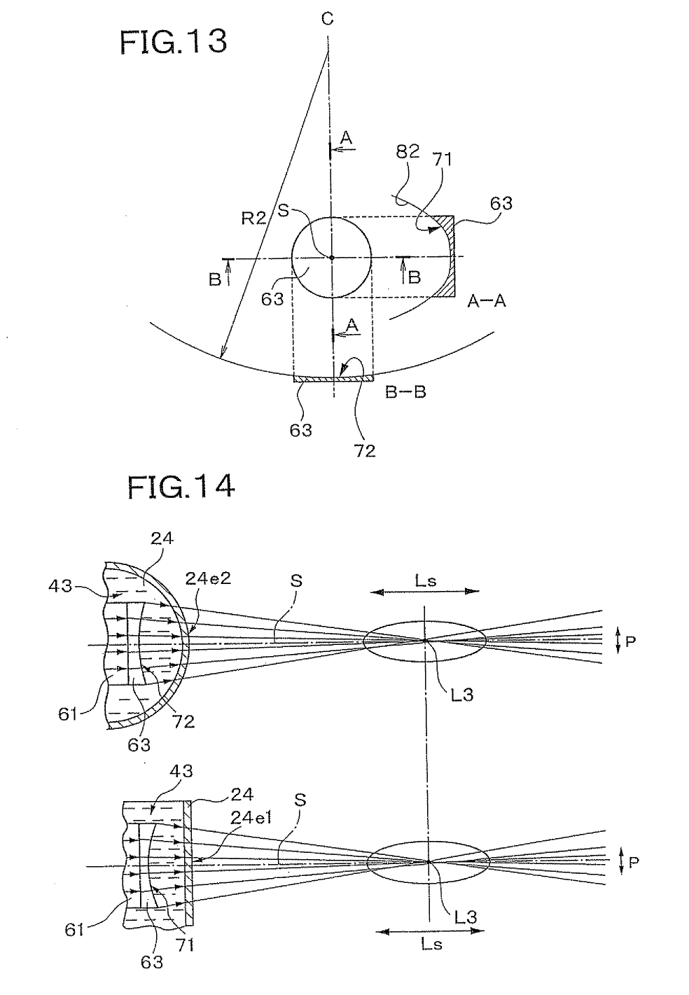

FIG. 13 is an enlarged perspective view of a lens used for explaining a method for defining a shape of the lens surface of the ultrasound transducer unit of the ultrasound transducer included by the ultrasound diagnostic apparatus showing the present embodiment;

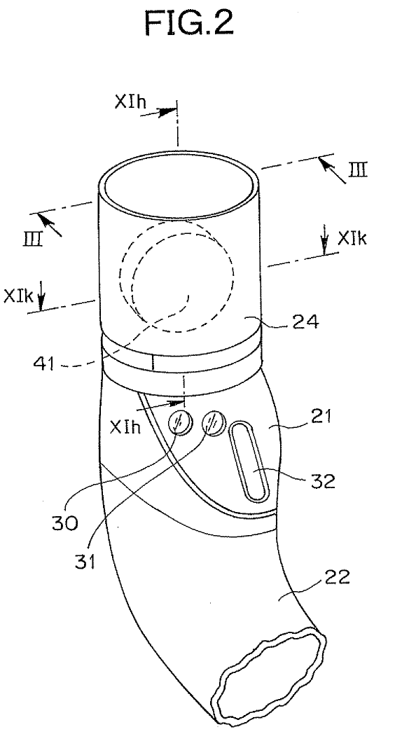

FIG. 19 is a plane view showing a modified example in which a lens of the ultrasound transducer in FIG. 2 is formed into a doughnut shape with a cross-sectional view taken along line XIX-XIX; and

FIG. 20 is a perspective view showing a modified example in which the cap in FIG. 2 is formed into a barrel shape.

### DETAILED DESCRIPTION OF THE PREFERRED EMBODIMENTS

Hereinafter, embodiments of the present invention will be described with reference to the drawings. In the present embodiments which will be described hereinafter, explanation will be made by citing the case in which an ultrasound diagnostic apparatus is applied to an ultrasound endoscope as an example.

### First Embodiment

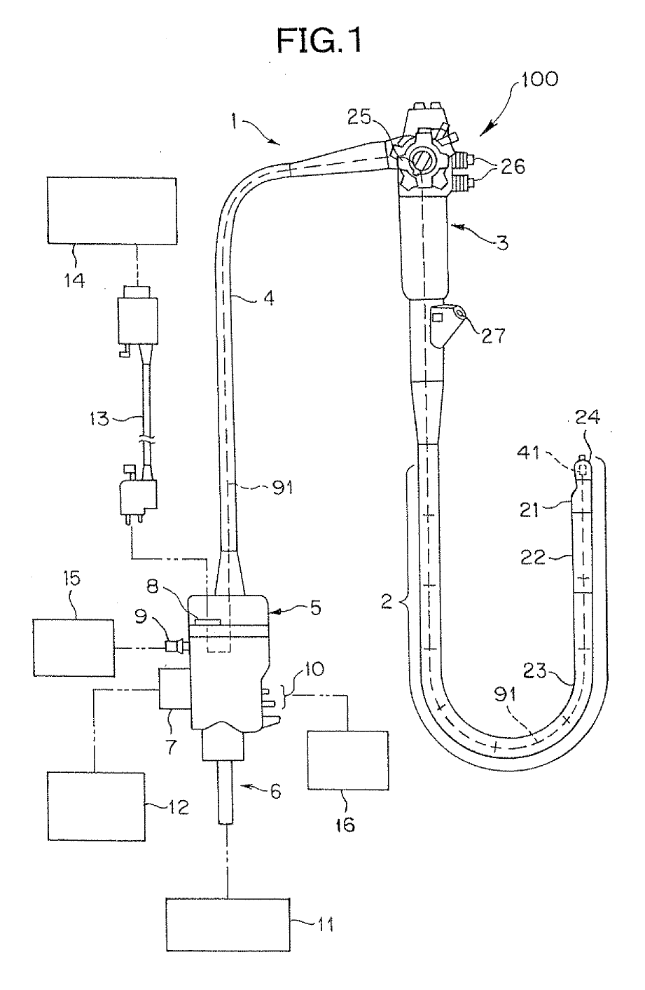

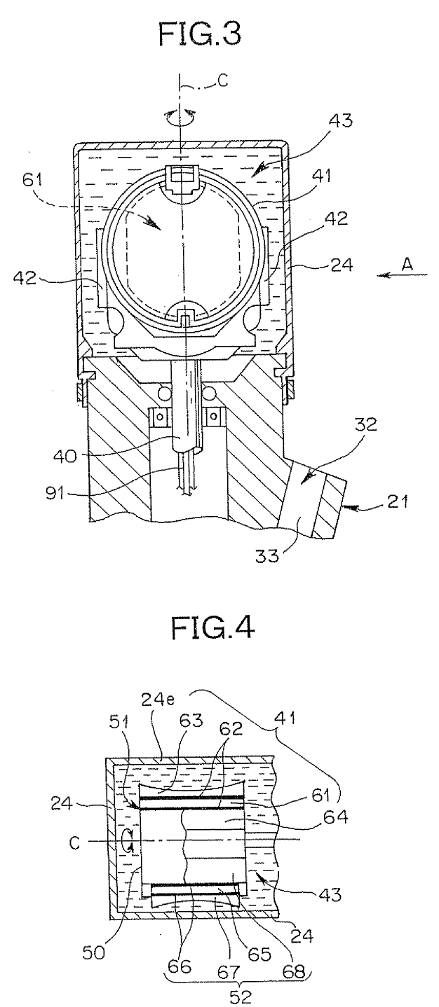

FIG. 1 is a view showing a configuration of an ultrasound endoscope apparatus including an ultrasound diagnostic apparatus of the present embodiment, FIG. 2 is a perspective view schematically showing in enlarged manner a distal end portion of an insertion portion of an ultrasound endoscope of FIG. 1, FIG. 3 is a partial cross-sectional view taken along line III-III in FIG. 2 showing an ultrasound transducer to be seen from a lens surface side, and FIG. 4 is a partial cross-sectional view showing a configuration of the ultrasound transducer in FIG. 3 with a cap from a direction differing from FIG. 3 by 90 degrees (direction A in FIG. 3).

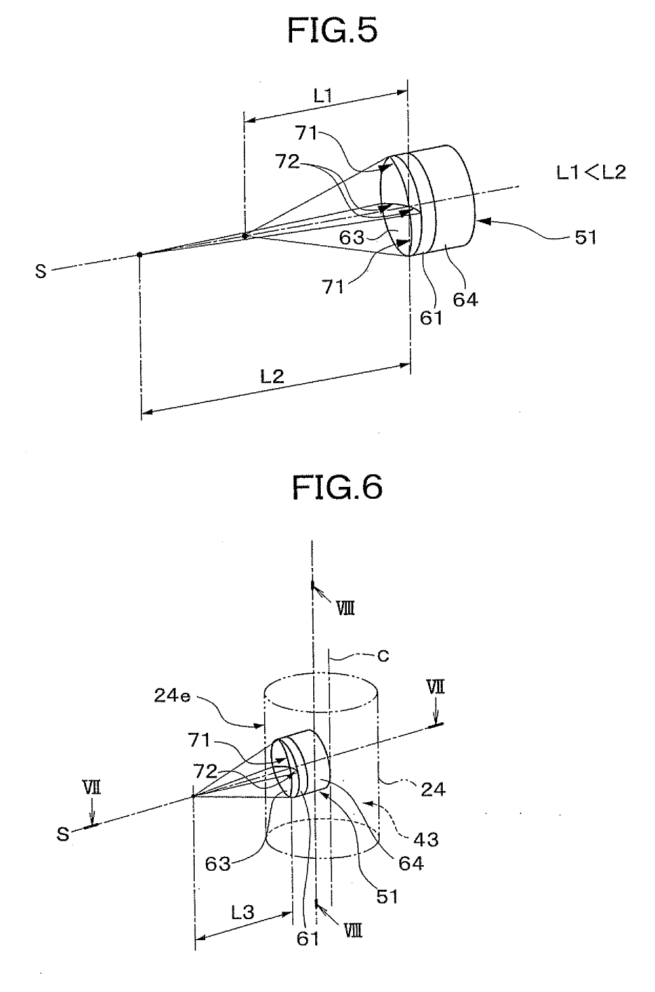

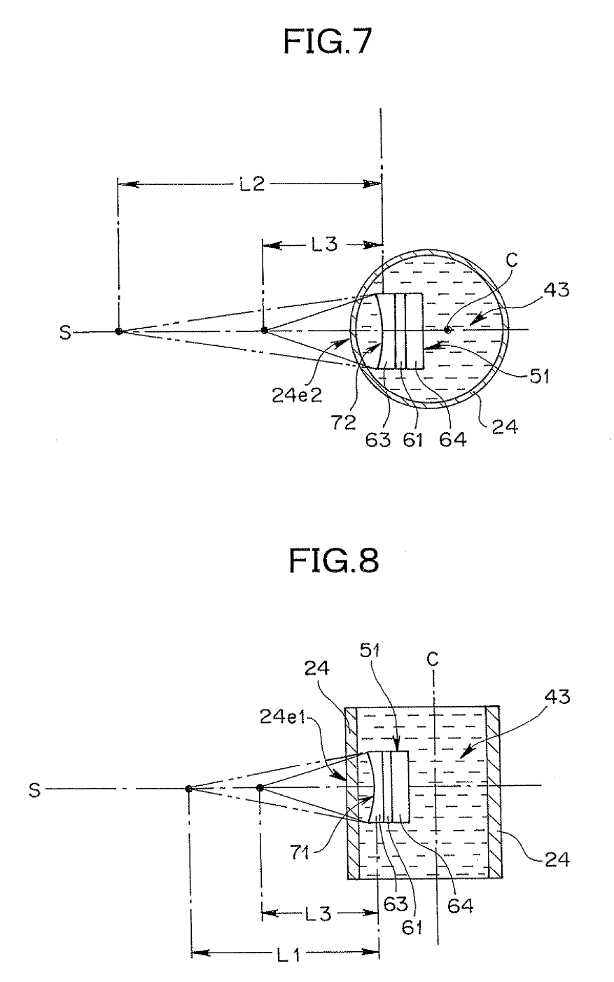

FIG. 5 is a perspective view showing focal lengths of a first lens surface and a second lens surface in a lens of an ultrasound transducer unit in FIG. 4, FIG. 6 is a perspective view showing a state in which the focal lengths of the first lens surface and the second lens surface correspond to each other in a state in which the ultrasound transducer unit in FIG. 5 is provided in the cap with an acoustic medium, FIG. 7 is a cross-sectional view taken along line VII-VII in FIG. 6, and FIG. 8 is a sectional view taken along line VIII-VIII in FIG. 6.

Hereinafter, the configuration of the ultrasound endoscope shown in FIGS. 1 to 7 will be omitted or schematically described except for a main part for simplifying the explanation.

As shown in FIG. 1, in an ultrasound endoscope apparatus 100, a main part is configured by including an ultrasound endoscope 1, a light source device 11, a video processor 12, an ultrasound observation device 14, a suction pump 15 and a water supply tank 16.

In the ultrasound endoscope 1, a main part is configured by an elongated insertion portion 2 which is inserted into a cavity, an operation portion 3 which is provided at a proximal end portion in an insertion direction of the insertion portion 2 and is also used as a grasping portion, a universal cord 4 which is extended from, for example, a proximal end side in the insertion direction and has flexibility, and a scope connector 5 provided at an extension end portion of the universal cord 4.

The scope connector 5 is provided with a light source connector 6, an electric connector 7, an ultrasound connector 8, a suction mouthpiece 9 and an air supply and water supply mouthpiece 10.

The light source device 11 which supplies illumination light is configured to be attachable to and detachable from the light source connector 6. The video processor 12 which performs various kinds of signal processing and the like via a signal cable (not illustrated) is configured to be attachable to and detachable from the electric connector 7.

Further, the ultrasound observation device 14 is configured to be attachable to and detachable from the ultrasound connector 8 via an ultrasound cable 13. The suction pump 15 is configured to be attachable to and detachable from the suction mouthpiece 9 via a suction tube (not illustrated). The water supply tank 16 is configured to be attachable to and detachable from the air supply and water supply mouthpiece 10 via an air supply/water supply tube not illustrated.

The ultrasound observation device 14 performs control of various operations of the ultrasound endoscope 1, and performs drive control of an ultrasound transducer 41 which will be described later, for example, and an operation of generating a video signal by performing signal processing of an electric signal obtained by the drive control of the ultrasound transducer 41.

A video signal generated in the ultrasound observation device 14 is outputted to a display device (not illustrated) which configures the ultrasound endoscope apparatus. As a result, an ultrasound image is displayed on a screen of the display device which receives the video signal The ultrasound observation device 14 configures a part of the ultrasound diagnostic apparatus.

The insertion portion 2 of the ultrasound endoscope 1 is configured by connectively providing a distal end rigid portion 21 formed of a rigid member, a bending portion 22 configured to be bendable in a vertical direction and a lateral direction, for example, and a flexible tube portion 23 which is long and has flexibility, in order from a distal end side.

The operation portion 3 is provided with a bending operation knob 25 which performs a bending operation of the bending portion 22, and an air supply and water supply suction buttons 26 which respectively perform an air supply and water supply operation and a suction operation. At a side of the insertion portion 2 of the operation portion 3, a treatment instrument insertion port 27 for introducing a treatment instrument into a cavity via a treatment instrument insertion tube path 33 provided inside the insertion portion 2 and the operation portion 3 (see FIG. 3) is provided.

As shown in FIG. 2, a lens for illumination 30, a lens for observation 31 and an opening for a treatment instrument 32 are provided at set positions on an outer peripheral side surface of the distal end rigid portion 21.

The lens for illumination 30 emits illumination light supplied from the light source device 11 toward an observation part to be a subject part in a cavity through a light guide (not illustrated) which is inserted through insides of the universal cord 4, the operation portion 3 and the insertion portion 2. The observation part in the cavity is illuminated with the illumination light emitted from the lens for illumination 30.

The lens for observation 31 configures an observation unit together with a plurality of objective lenses (not illustrated) provided in the distal end rigid portion 21, and an image pickup device (not illustrated) such as a charge coupled device provided at an image forming position of the objective lenses.

An optical image of the observation part observed by the lens for observation 31 and the objective lens is formed on an image pickup surface of the image pickup device, and thereafter, is subjected to photoelectric conversion to be an electric signal in the image pickup device. Thereafter, the electric signal is transmitted to the video processor 12 via an image pickup cable (not illustrated) which is extended from the image pickup device and inserted through-the insides of the insertion portion 2, the operation portion 3 and the universal cord 4 and the electric connector 7.

The video processor 12 generates a standard video signal by performing signal processing for the transmitted electric signal, and outputs the video signal to the display device not illustrated. As a result, an endoscope observation image is displayed on the screen of the display device.

The opening for a treatment instrument 32 is an opening at the distal end rigid portion 21 side, of the treatment instrument insertion tube path 33 provided in the insertion portion 2 and the operation portion 3 (see FIG. 3), and is an opening for protruding the treatment instrument which is inserted into the treatment instrument insertion tube path 33 from the treatment instrument insertion port 27 to the subject part in the cavity.

A cap 24, for example, in a cylindrical shape configured by, for example, polyethylene is provided on a distal end side in the insertion direction from the lens for illumination 30, the lens for observation 31 and the opening for a treatment instrument 32 of the distal end rigid portion 21, as shown in FIG. 2. The ultrasound transducer 41 as well as an acoustic medium 43 is provided in the cap 24 as shown in FIG. 3. The cap 24, the acoustic medium 43 and the ultrasound transducer 41 configure a part of the ultrasound diagnostic apparatus.

An opening at a distal end of the cap 24 in the insertion direction is closed. The insertion portion 2 of the ultrasound endoscope 1 is used by being passed through the throat part of a subject, and therefore, the diameter of the cap 24 is preferably formed to be as small as possible, for example, 20 mm or less.

Further, when the insertion portion 2 is inserted into the cavity, the cap 24 contacts a cavity wall in the cavity via a medium outside the cap such as water and body fluid in which the sound velocity at which ultrasound passes is about 1500 m/sec.

The cap 24 is placed so that an ultrasound transducer rotational axis (swing axis) C and a cylinder center of the cap 24 correspond to each other.

The ultrasound transducer 41 is formed into a substantially column-shaped member as will be described later, and is provided in the cap 24 with its outer periphery held by a pair of ultrasound transducer holders 42 provided in the distal end rigid portion 21, and each having a concave portion projected to the distal end side in the insertion direction.

The holding diameter of the pair of ultrasound transducer holders 42 is formed to be smaller than an outside diameter of the ultrasound transducer 41. Thereby, the pair of ultrasound transducer holders 42 holds the ultrasound transducer 41 with predetermined contact pressure.

The ultrasound transducer holders 42 are connected to a rotary shaft 40 rotatable with the ultrasound transducer rotational axis C as a center of rotation by rotating means such as a motor not illustrated. Thereby, the ultrasound transducer holders 42 rotate (swing) with the ultrasound transducer rotational axis C as the center of rotation.

As a result, the ultrasound transducer 41 held by the ultrasound transducer holders 42 is rotatable in the cap 24. The ultrasound transducer 41 is held by the ultrasound transducer holders 42 so that a distance between an outer peripheral surface of the ultrasound transducer 41 and an inner peripheral surface of the cap 24 becomes constant irrespective of rotation.

The rotary shaft 40 is configured by a cylindrical member, and inside the rotary shaft 40, a signal cable 91 which is connected to the ultrasound connector 8 via the insides of the insertion portion 2, the operation portion 3 and the universal cord 4 is extended from the ultrasound transducer 41 as shown in FIG. 1.

The ultrasound transducer 41 includes, for example, two ultrasound transducer units 51 and 52 as shown in FIG. 4, and the two ultrasound transducer units 51 and 52 are disposed to be opposed with respect to the ultrasound transducer rotational axis C and held by a housing 50 configuring a cylindrical casing of the ultrasound transducer 41.

An outer shape of the ultrasound transducer unit 52 is formed smaller than that of the ultrasound transducer unit 51. The ultrasound transducer 41 may include only any one of the ultrasound transducer units 51 and 52.

In the ultrasound transducer unit 51, a main part is configured by a piezoelectric element 61 which is an electromechanical transducer element configured by, for example, ceramics, electrodes 62 which are provided thinly on one surface of the piezoelectric element 61 and on the other surface opposed to the one surface and configured by any one of Ni, gold, silver and the like, a concave lens 63 which is provided on a side of the electrode 62 on the one surface of the piezoelectric element 61 and configured by, for example, an epoxy resin, and a backing material 64 which is provided on a side of the electrode 62 on the other surface of the piezoelectric element 61 and configured by, for example, rubber.

The piezoelectric element 61 is configured by a plate-shaped piezoelectric sensor which transmits the ultrasound of frequencies of, for example, 5.0 MHz and 7.5 MHz. The electrodes 62 are formed on the one surface and the other surface of the piezoelectric element 61 by being plated, for example. The electrode 62 has the functions of deforming particles of the ceramics configuring the piezoelectric element 61 by applying voltage to the piezoelectric element 61, transmitting ultrasound by using the piezoelectric element 61 as a sound source, and receiving a potential difference from deformation of the piezoelectric element 61 after receiving the ultrasound reflecting from a subject part after the transmission.

The concave lens 63 has the function of transmitting the ultrasound transmitted by using one surface of the piezoelectric element 61 as the sound source by bringing the ultrasound into focus on the subject part so as not to be diffused. The backing material 64 has the function of attenuating the ultrasound transmitted by using the other surface of the piezoelectric element 61 as the sound source.

In the ultrasound transducer unit 52, a main part is configured by a piezoelectric element 65 which is an electromechanical transducer element configured by, for example, ceramics, electrodes 66 which are provided thinly on one surface of the piezoelectric element 65 and on the other surface opposed to the one surface and configured by any one of Ni, gold, silver and the like, a concave lens 67 which is provided on a side of the electrode 66 on the one surface of the piezoelectric element 65 and configured by, for example, an epoxy resin, and a backing material 68 which is provided on a side of the electrode 66 of the other surface of the piezoelectric element 65 and configured by, for example, rubber.

The piezoelectric element 65 is configured by a plate-shaped piezoelectric sensor which transmits the ultrasound of frequencies higher than those of the piezoelectric element 61, for example, frequencies of 20 MHz and 12 MHz. An outer shape of the piezoelectric element 65 is formed to be smaller than that of the above described piezoelectric element 61.

The electrode 66, the concave lens 67 and the backing material 68 have the equivalent functions to those of the above described electrode 62, the concave lens 63 and the backing material 64, and therefore, explanation thereof will be omitted, but their outer shapes are formed to be smaller than those of the electrode 62, the concave lens 63 and the backing material 64. The electrodes 66 are formed on the one surface and the other surface of the piezoelectric element 65 by being plated, for example.

The ultrasound transducer 41 transmits ultrasound to a subject part via a cylinder surface 24e of the cap 24, receives the ultrasound reflecting from the subject part after transmission and converts the ultrasound into an electric signal, and thereafter, transmits the electric signal to the ultrasound observation device 14 via the signal cable 91, the ultrasound connector 8 and the ultrasound cable 13. The signal cable 91, the ultrasound connector 8 and the ultrasound cable 13 configure a part of the ultrasound diagnostic apparatus.

The ultrasound observation device 14 performs signal processing for the transmitted electric signal, thereby, generates an ultrasound image signal, and outputs the ultrasound image signal to the display device not illustrated. As a result, an ultrasound image is displayed on the screen of the display device.

The ultrasound transducer 41 includes the two ultrasound transducer units 51 and 52 including the piezoelectric elements 61 and 65 differing in frequency as described above. Therefore, any one of the ultrasound transducer units 51 and 52 is opposed to a subject part by rotating the ultrasound transducer 41, and ultrasound is transmitted to and received from the subject part from and to any one of the ultrasound transducer units 51 and 52 via the cylinder surface 24e of the cap 24, whereby favorable ultrasound images of subject parts at different distances from the ultrasound transducer 41 can be obtained.

Specifically, the ultrasound transducer unit 52 including the piezoelectric element 65 configured by the piezoelectric sensor having frequencies of 20 MHz and 12 MHz is opposed to a subject part by rotation to transmit and receive ultrasound, and thereby, a favorable ultrasound image of the subject part adjacent to the ultrasound transducer 41 can be obtained via the cylinder surface 24e.

Meanwhile, the ultrasound transducer unit 51 including the piezoelectric element 61 configured by the piezoelectric sensor having frequencies of 5.0 MHz and 7.5 MHz is opposed to a subject part by rotation to transmit and receive ultrasound, and thereby, a favorable ultrasound image of the subject part away from the ultrasound transducer 41 can be obtained via the cylinder surface 24e.

The acoustic medium 43 is configured by an oil medium such as glycerin in which the sound velocity at which ultrasound passes is lower than in the above described medium outside the cap, for the purpose of enhancing smoothness of rotation of the ultrasound transducer 41 and preventing bubbles which occur in the acoustic medium 43 with the rotation of the ultrasound transducer 41, and is filled between the inner periphery surface of the cap 24 and the ultrasound transducer 41 inside the cap 24.

Here, each of the lenses 63 and 67 of the ultrasound transducer units 51 and 52 in the ultrasound transducer 41 is formed so that a first focal length L1, as shown in FIGS. 5 and 8, of a first lens surface 71 on a section parallel with the ultrasound transducer rotational axis C including a center of a sound beam axis S in each of single bodies of the lenses 63 and 67 is shorter than a second focal length L2, as shown in FIGS. 5 and 7, of a second lens surface 72 on a section orthogonal to the ultrasound transducer rotational axis C including the center of the sound beam axis S in each of the lenses 63 and 67 (L1<L2). FIGS. 5 to 8 are the drawings illustrating only the ultrasound transducer unit 51 of the ultrasound transducer 41 for simplifying the drawings.

Here, the medium outside the cap 24 in the cavity is water, body fluid and the like in which the velocity at which ultrasound passes is higher than in the oil medium, and therefore, when an oil medium is used for the acoustic medium 43 as in the present embodiment, the effect of a pseudo lens in which the refraction direction of ultrasound changes occurs in the acoustic medium 43 and the cylinder surface 24e of the cap 24 (see FIG. 6) as the sound velocity at which the ultrasound transmitted from the ultrasound transducer 41 passes changes outside and inside of the cap

More specifically, comparing first ultrasound transmitted from the first lens surface 71 and second ultrasound transmitted from the second lens surface 72 in the lens 63, the first ultrasound passes through a cylinder surface 24e1 with a small curvature parallel with the ultrasound transducer rotational axis C of the cap 24 in a substantially straight line as shown in FIG. 8, whereas the second ultrasound refractively passes through a cylinder surface 24e2 having a large curvature, for example, the cylinder surface 24e2 having a curvature of a radius of 10 mm when the diameter of the cap 24 is 20 mm, by the known Snell's law as shown in FIG. 7. This causes the problem that the focal positions of each of ultrasound significantly differ on the first lens surface 71 and the second lens surface 72 in the lens 63 due to difference in the refraction direction of ultrasound, and a favorable ultrasound image cannot be obtained.

However, the experimental result shows that the first focal length L1 of the first lens surface 71 is set to be shorter than the second focal length L2 of the second lens surface 72 in advance as shown in FIG. 5 as in the present embodiment, whereby even if the acoustic medium 43 composed of an oil medium and the cylinder surface 24e of the cap 24 cause the pseudo lens effect when the ultrasound transducer 41 is provided in the cap 24 together with the acoustic medium 43 as shown in FIG. 6, the focal positions of the first lens surface 71 and the second lens surface 72 correspond to each other at a third focal position L3, or a deviation of the focal position of the first lens surface 71 can be suppressed to the minimum deviation, that is, within 3 to 6% when the focal point of the second lens surface 72 is set as a reference, for example, as shown in FIGS. 6 to 8.

The above similarly applies to the lens 67 in the ultrasound transducer unit 52. As above, in the present embodiment, the lenses 63 and 67 are formed in advance so that the focal length L1 of the first lens surface 71 becomes shorter than the focal length L2 of the second lens surface 72.

According to the above, even if the acoustic medium 43 composed of an oil medium in which the sound velocity at which ultrasound passes is lower than in the medium outside the cap, and the cylinder surface 24e of the cap 24 cause a pseudo lens effect, the focal positions of the first lens surface 71 and the second lens surface 72 correspond to each other in the third focal position L3 or have the minimum deviation, and therefore, the ultrasound diagnostic apparatus capable of obtaining a favorable ultrasound image can be provided.

### Second Embodiment

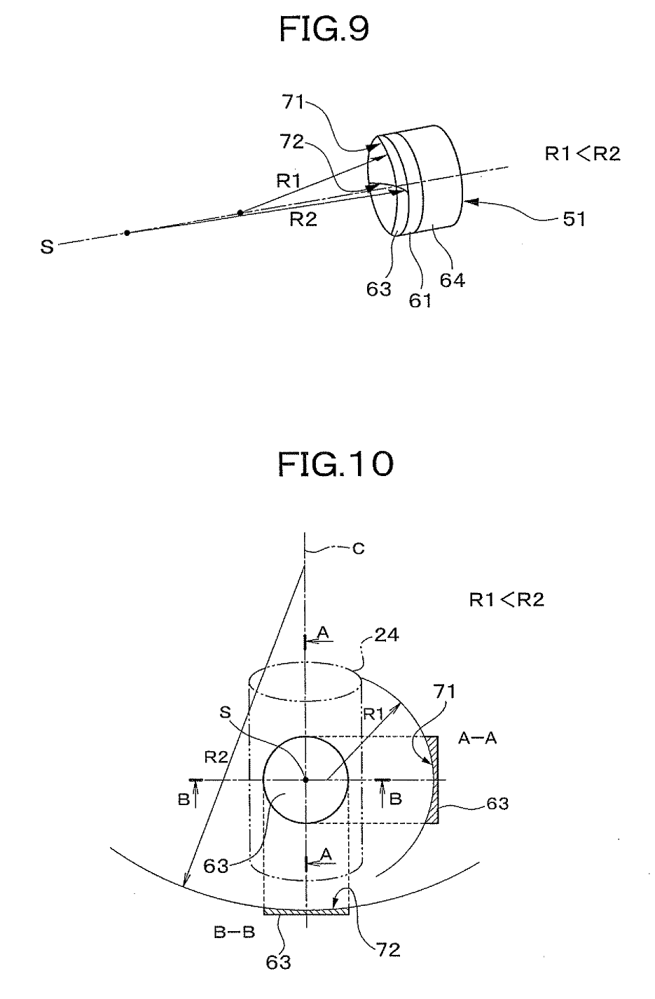

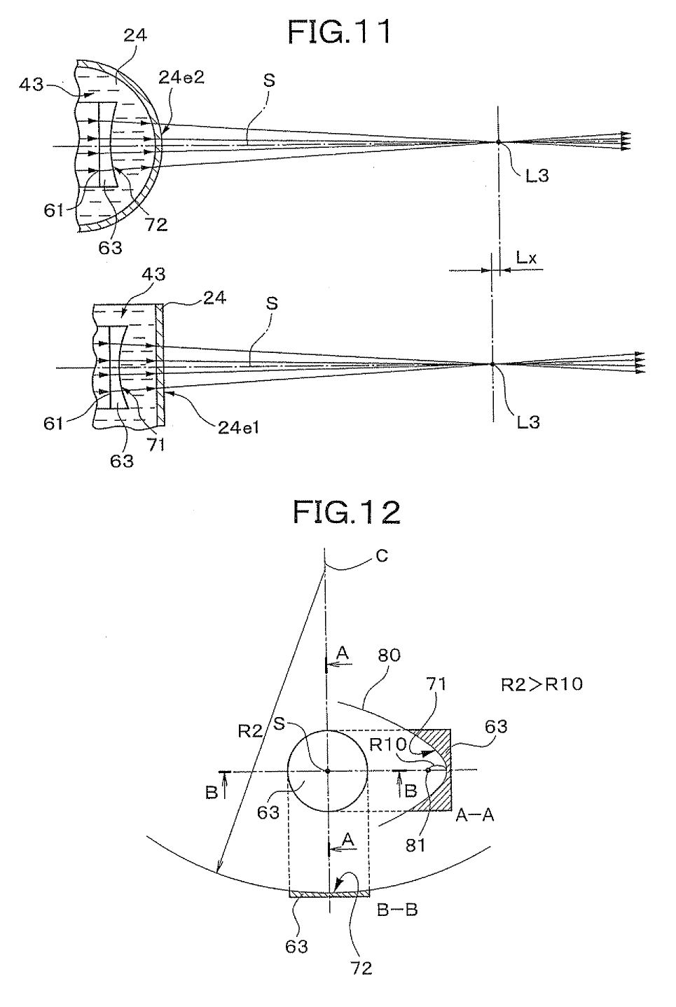

FIG. 9 is a perspective view showing a configuration of a ultrasound transducer unit of an ultrasound transducer included by the ultrasound diagnostic apparatus showing the present embodiment, FIG. 10 is a view showing a lens shape in FIG. 9 with a section of the first lens surface and a section of the second lens surface, and FIG. 11 is a view showing a partial cross-sectional view taken along line XIk-XIk in FIG. 2 with a partial cross-sectional view taken along line XIk-XIk in FIG. 2.

In the present embodiment, a lens shape of the above described first embodiment for setting the focal length L1 of the first lens surface 71 to be shorter than the focal length L2 of the second lens surface 72 will be described hereinafter. Therefore, the same components as those of the first embodiment are assigned with the same reference numerals and characters, and explanation thereof will be omitted.

In the present embodiment, the drawings in which only the ultrasound transducer unit 51 of the ultrasound transducer 41 is illustrated are also adopted for simplifying the drawings.

As shown in FIGS. 9 and 10, in the lens 63 in the present embodiment, the first lens surface 71 and the second lens surface 72 are formed into partial circular-arc shapes, and a first radius of curvature R1 in an A-A section of the first lens surface 71 is formed to be smaller than a second radius of curvature R2 in a B-B section of the second lens surface 72 (R1<L).

The experiment result shows that according to such a configuration of the lens 63, the radius of curvature R1 of the first lens surface 71 is formed to be smaller than the radius of curvature R2 of the second lens surface 72 (R1<R2), and thereby, the focal length L1 of the first lens surface 71 can be set to be shorter than the focal length L2 of the second lens surface 72 as described above (L1<L2).

As a result, even if the acoustic medium 43 composed of an oil medium in which the sound velocity at which ultrasound passes is lower than in the medium outside the cap and the cylinder surface 24e of the cap 24 cause a pseudo lens effect, the focal positions of the first lens surface 71 and the second lens surface 72 correspond to each other in the third focal position L3 or have a minimum deviation Lx as shown in FIG. 11, and therefore, the ultrasound diagnostic apparatus capable of obtaining a favorable ultrasound image can be provided.

The above description similarly applies to the lens 67 of the ultrasound transducer unit 52.

### Third Embodiment

FIG. 12 is a view showing a lens shape differing from that in FIG. 10 of the ultrasound transducer unit of the ultrasound transducer included by the ultrasound diagnostic apparatus showing the present embodiment with the section of the first lens surface and the section of the second lens surface, FIG. 13 is a view showing a lens shape differing from that of FIG. 12 with the section of the first lens surface and the section of the second lens surface, and FIG. 14 is a partial cross-sectional view showing focal positions of the first lens surface and the second lens surface in the present embodiment.

In the present embodiment, a lens shape of the above described first embodiment, which is for setting the focal length L1 of the first lens surface 71 to be shorter than the focal length L2 of the second lens surface 72, and is different from that of the second embodiment, will be described hereinafter. Therefore, the same components as those of the first embodiment are assigned with the same reference numerals and characters, and explanation thereof will be omitted.

In the present embodiment, the drawings in which only the ultrasound transducer unit 51 of the ultrasound transducer 41 is illustrated are also adopted for simplification of the drawings.

As shown in FIG. 12, in the lens 63 in the present embodiment, the first lens surface 71 in the A-A section is formed into a shape defined by an absolute value of a parabola 80 of the second degree, and the second lens surface 72 in the B-B section is formed into a partial circular-arc shape. A focal length R10 from a parabola focal point 81 of the parabola 80 is formed to be smaller than the radius of curvature R2 in the B -B section of the second lens surface 72 (R10<R2).

As shown in FIG. 13, the first lens surface 71 in the A-A section may be formed into a shape defined by an absolute value of a parabola 82 of the third degree. Though not illustrated, the first lens surface 71 in the A-A section may be formed into a shape defined by an absolute value of a cycloid curve.

The experimental result shows that according to such a configuration of the lens 63, the focal length L1 of the first lens surface 71 can be set to be shorter than the focal length L2 of the second lens surface 72 as described above (L1<L2).

As a result, even if the acoustic medium 43 composed of an oil medium in which the sound velocity at which ultrasound passes is lower than in the medium outside the cap and the cylinder surface 24e of the cap 24 cause a pseudo lens effect, the focal positions of the first lens surface 71 and the second lens surface 72 correspond to each other in the third focal position L3 or have the minimum deviation, and therefore, the ultrasound diagnostic apparatus capable of obtaining a favorable ultrasound image can be provided.

Further, the experimental result shows that according to the shape of the lens 63 in the present embodiment, a region P which is brought into focus can be set to be longer as Ls than the conventional lens in the third focal position L3 as shown in FIG. 14, though the region which is brought into focus is only a limited region in the third focal position L3 in the above described first and the second embodiments.

Therefore, the ultrasound diagnostic apparatus capable of obtaining a more favorable ultrasound image than in the above described second embodiment can be provided.

The above description similarly applies to the lens 67 of the ultrasound transducer unit 52.

### Fourth Embodiment

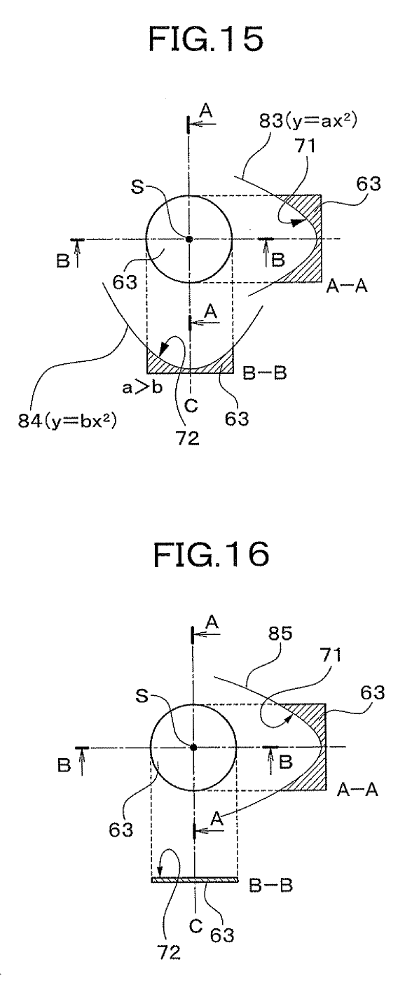

FIG. 15 is a view showing a lens shape differing from those in FIGS. 10 and 12 of the ultrasound transducer unit of the ultrasound transducer included by the ultrasound diagnostic apparatus showing the present embodiment with the section of the first lens surface and the section of the second lens surface.

In the present embodiment, a lens shape of the above described first embodiment, which is for setting the focal length L1 of the first lens surface 71 to be shorter than the focal length L2 of the second lens surface 72, and is different from those of the second and the third embodiments, will be described hereinafter. Therefore, the same components as those of the first embodiment are assigned with the same reference numerals and characters, and explanation thereof will be omitted.

In the present embodiment, the drawing in which only the ultrasound transducer unit 51 of the ultrasound transducer 41 is illustrated is also adopted for simplification of the drawing.

As shown in FIG. 15, in the lens 63 in the present embodiment, the first lens surface 71 in the A-A section is formed into a shape defined by an absolute value of a parabola 83 or curve of third or higher order, and the second lens surface 72 in the B-B section is formed into a shape defined by an absolute value of a parabola 84 or curve of third or higher order differing from the parabola 83 in coefficient.

More specifically, when the parabolas 83 and 84 are set as parabolas of the second degree, the first lens surface 71 in the A-A section is formed into the shape defined by an absolute value of the parabola 83 of y=ax², and the second lens surface 72 in the B-B section is formed into the shape defined by an absolute of the parabola 84 of y=bx² where a second coefficient b is smaller than a first coefficient a(b<a).

The experimental result shows that according to such a configuration of the lens 63, the focal length L1 of the first lens surface 71 can be also set to be shorter than the focal length L2 of the second lens surface 72 as described above (L1<L2).

As a result, even if the acoustic medium 43 composed of an oil medium in which the sound velocity at which ultrasound passes is lower than in the medium outside the cap, and the cylinder surface 24e of the cap 24 cause a pseudo lens effect, the focal positions of the first lens surface 71 and the second lens surface 72 correspond to each other in the third focal position L3 or have the minimum deviation, and therefore, the ultrasound diagnostic apparatus capable of obtaining a favorable ultrasound image can be provided.

Further, the experimental result shows that according to the shape of the lens 63 in the present embodiment, the above described region P which is brought into focus can be set to be. Ls or more and longer in the third focal position L3, than in the above described second embodiment, than especially in the case in which the first lens surface 71 in the A-A section is formed into the shape defined by the absolute value of the parabola 82 of the third degree as shown in the above described FIG. 13.

Therefore, the ultrasound diagnostic apparatus capable of obtaining more favorable ultrasound image than in the above described second and third embodiments can be provided.

The above description similarly applies to the lens 67 of the ultrasound transducer unit 52.

### Fifth Embodiment

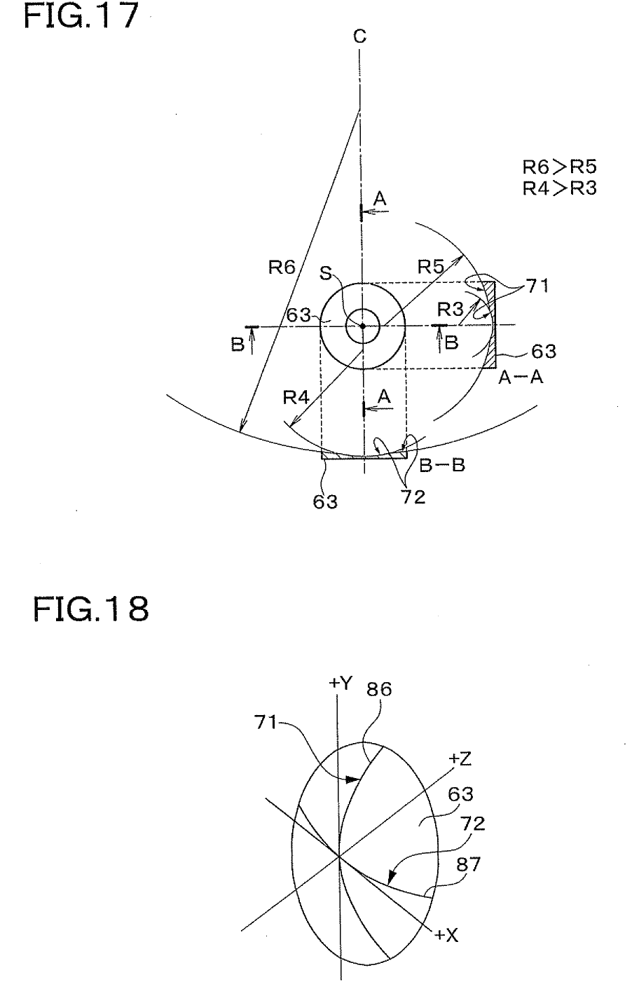

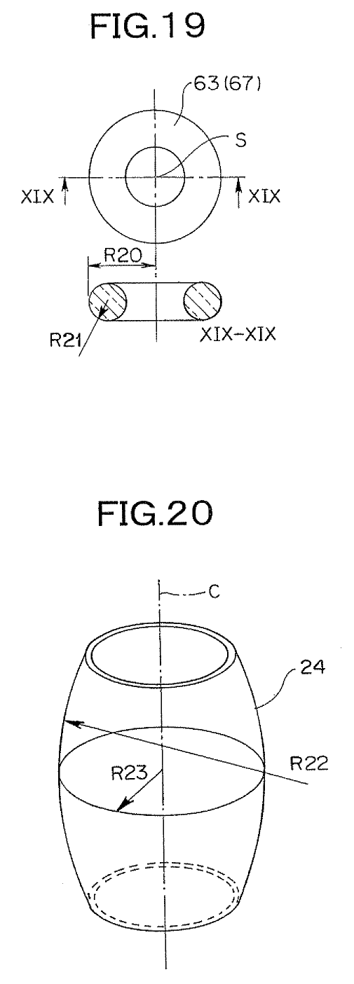

FIG. 16 is a view showing a lens shape differing from those in FIGS. 10, 12 and 15 of the ultrasound transducer unit of the ultrasound transducer included by the ultrasound diagnostic apparatus showing the present embodiment with the section of the first lens surface and the section of the second lens surface.

In the present embodiment, a lens shape of the above described first embodiment, which is for setting the focal length L1 of the first lens surface 71 to be shorter than the focal length L2 of the second lens surface 72, and is different from those of the second to the fourth embodiments, will be described hereinafter. Therefore, the same components as those of the first embodiment are assigned with the same reference numerals and characters, and explanation thereof will be omitted.

In the present embodiment, the drawing in which only the ultrasound transducer unit 51 of the ultrasound transducer 41 is illustrated is also adopted for simplification of the drawing.

As shown in FIG. 16, in the lens 63 of the present embodiment, the first lens surface 71 in the A-A section is formed into a shape defined by an absolute value of a parabola 85 or curve of third or higher order, and the second lens surface 72 in the B-B section is formed into a plane shape.

Specifically, the second lens surface 72 is formed into the shape which does not have a lens effect. Thereby, in a direction orthogonal to the ultrasound transducer rotational axis C including the center of the sound beam axis S in the lens 63, the pseudo lens configured by the acoustic medium 43 and the cylinder surface 24e2 of the cap 24 function to bring ultrasound into focus on the third focal position L3.

The experimental result shows that according to such a configuration of the lens 63, the focal length L1 of the first lens surface 71 can be also set to be shorter than the focal length L2 of the second lens surface 72 as described above (L1<L2).

As a result, even if the acoustic medium 43 composed of an oil medium in which the sound velocity at which ultrasound passes is lower than in the medium outside the cap, and the cylinder surface 24e of the cap 24 cause a pseudo lens effect, the focal positions of the first lens surface 71 and the second lens surface 72 correspond to each other in the third focal position L3 or have the minimum deviation, and therefore, the ultrasound diagnostic apparatus capable of obtaining a favorable ultrasound image can be provided.

Further, according to the shape of the lens 63 in the present embodiment, the second lens surface 72 is formed into the shape which does not have a lens effect, and therefore, machining of the lens 63 can be made easier than in the above described second to fourth embodiments.

The above description similarly applies to the lens 67 of the ultrasound transducer unit 52.

### Sixth Embodiment

FIG. 17 is a view showing a lens shape differing from those of FIGS. 10, 12, 15 and 16 of the ultrasound transducer unit of the ultrasound transducer included by the ultrasound diagnostic apparatus showing the present embodiment with the section of the first lens surface and the section of the second lens surface.

In the present embodiment, a lens shape of the above described first embodiment, which is for setting the focal length L1 of the first lens surface 71 to be shorter than the focal length L2 of the second lens surface 72, and is different from those of the second to the fifth embodiments, will be described hereinafter. Therefore, the same components as those of the first embodiment are assigned with the same reference numerals and characters, and explanation thereof will be omitted.

In the present embodiment, the drawing in which only the ultrasound transducer unit 51 of the ultrasound transducer 41 is illustrated is also adopted for simplification of the drawing.

As shown in FIG. 17, in the lens 63 of the present embodiment, the first lens surface 71 in the A-A section and the second lens surface 72 in the B-B section are formed into partial circular-arc shapes. In more detail, the first lens surface 71 is formed into a shape with multiple steps from a surface having two radiuses of curvature including a third radius of curvature R3 and a fourth radius of curvature R5 in the A-A section of the first lens surface 71. The second lens surface 72 is formed into a shape with multiple steps from a surface having two radiuses of curvature including a fifth radius of curvature R4 which is larger than the third radius of curvature R3, and a sixth radius of curvature R6 which is larger than the fourth radius of curvature R5 and differs from the fifth radius of curvature R4, in the B-B section of the second lens surface 72.

The experimental result shows that according to such a configuration of the lens 63, the focal length L1 of the first lens surface 71 can be also set to be shorter than the focal length L2 of the second lens surface 72 as described above (L1<L2).

As a result, even if the acoustic medium 43 composed of an oil medium in which the sound velocity at which ultrasound passes is lower than in the medium outside the cap, and the cylinder surface 24e of the cap 24 cause a pseudo lens effect, the focal positions of the first lens surface 71 and the second lens surface 72 correspond to each other in the third focal position L3 or have the minimum deviation, and therefore, the ultrasound diagnostic apparatus capable of obtaining a favorable ultrasound image can be provided.

Further, the experimental result shows that according to the shape of the lens 63 in the present embodiment, the above described region P which is brought into focus can be set to be as long as Ls in the third focal position L3 as in the above described second embodiment.

The above description similarly applies to the lens 67 of the ultrasound transducer unit 52.

### Seventh Embodiment

FIG. 18 is an enlarged perspective view of a lens used for explaining a method for defining a shape of the lens surface of the ultrasound transducer unit of the ultrasound transducer included by the ultrasound diagnostic apparatus showing the present embodiment.

In the above described second to sixth embodiments, the shapes of the first lens surface 71 and the second lens surface 72 are described, and in the present embodiment, a method for defining the shape of one lens surface in the lens 63 will be described hereinafter. Therefore, the same components as those in the first embodiment are assigned with the same reference numerals and characters, and explanation thereof will be omitted.

In the present embodiment, the drawing in which only the ultrasound transducer unit 51 of the ultrasound transducer 41 is illustrated is also adopted for simplification of the drawing.

In the present embodiment, when the first lens surface 71 and the second lens surface 72 are set to be in partial circular-arc shapes for the lens 63, one lens surface of the lens 63 can be defined by adding a second curve formula 87 defining the second lens surface 72 to a first curve formula 86 defining the first lens surface 71.

More specifically, the shape of the first lens surface 71 in a Y-Z plane is defined by the first curve formula 86 of

Z=R1−\sqrt{√(R1²−Y²)}

, and the shape of the second lens surface 72 in an X-Z plane is defined as

Z=R2−\sqrt{√(R2²+X²)}.

As a result, for the lens 63, the second curve formula 87 is added to the first curve formula 86, and thereby, one surface is defined by

Z=R1−\sqrt{√(R1²−Y²)}+R2−\sqrt{√(R2²−X²)}.

Even when for the lens 63, the first lens surface 71 in the A-A section and the second lens surface 72 in the B-B section are set to be in the shapes defined by the absolute values of the parabolas or curve of third or higher order, one lens surface is defined by adding the second curve formula 87 defining the second lens surface 72 to the first curve formula 86 defining the first lens surface 71.

More specifically, the shape of the first lens surface 71 in the Y-Z plane is defined by the first curve formula 86 of

Z=aY²

,and the shape of the second lens surface 72 in the X-Z plane is defined by the second curve formula 87 of

Z=bX².

Thereby, the lens 63 has one surface defined by

Z=aY²+bX²,

by adding the second curve formula 87 to the first curve formula 86. The above description similarly applies to the lens 67 of the ultrasound transducer unit 52.

Hereinafter, a modified example will be described by using FIG. 19. FIG. 19 is a plane view showing a modified example in which a lens of the ultrasound transducer in FIG. 2 is formed into a ring shape with a cross-sectional view taken along line XIX-XIX. The above described lenses 63 and 67 are not limited to the curved surfaces defined by the above described curved formulae as long as the curved surfaces satisfy the lenses described in the above described first to sixth embodiments.

For example, as shown in FIG. 19, each of the lenses 63 and 67 may be formed into a ring shape in which a radius from a center to an outer peripheral edge is R20, and a radius of a XIX-XIX section of the lens in the direction of the center of the sound beam axis S is R21. In this case, the piezoelectric elements 61 and 65 are placed in positions except for a center of a doughnut shape, that is, in positions superimposed on the ring-shaped lenses 63 and 67 seen in plane view.

In the above described first to seventh embodiments, explanation is made by citing the example of the case in which the ultrasound diagnostic apparatus is applied to the ultrasound endoscope, but the ultrasound diagnostic apparatus may be applied to an ultrasound probe without being limited thereto, and the same effects as in the first to seventh embodiments can be naturally obtained even when it is applied to other instruments.

Further, a modified example will be described by using FIG. 20 hereinafter. FIG. 20 is a perspective view showing a modified example in which the cap in FIG. 2 is formed into a barrel shape.

In the above described first to seventh embodiments, the cap 24 is formed into the cylindrical shape.

The cap 24 may be formed into a barrel shape having a curvature R22 of a surface parallel with the ultrasound transducer rotational axis C and a curvature R23 of a surface orthogonal to the ultrasound transducer rotational axis C, which is different from the curvature R22, as shown in FIG. 20, without being limited to the above.

When the cap 24 is formed into a barrel shape like this, not only the same effects as in the above described first to seventh embodiments can be obtained, but also a curvature difference between the curved surface parallel with the ultrasound transducer rotational axis C of each of the lenses 63 and 67 and the curved surface perpendicular to the ultrasound transducer rotational axis C can be made small in the cylinder surface parallel with the ultrasound transducer rotational axis C and the cylinder surface perpendicular to the ultrasound transducer rotational axis C in the cap 24, and therefore, the allowable range of an error of the curved surface in forming each of the lenses 63 and 67 can be increased in the cap 24. Specifically, the lenses 63 and 67 come to be easily formed.

When the cap 24 is formed into a barrel shape, the cap 24 is easily brought into close contact with the cavity wall in a cavity when the insertion portion 2 of the ultrasound endoscope 1 is inserted into the cavity.

Further, when the lens surfaces of the lenses 63 and 64 are formed into spherical surfaces, substantially the same effects as in the above described embodiments can be obtained even if the cap 24 is also formed into a spherical shape. If the cap 24 is formed into a spherical shape like this, formation of the cap 24 becomes facilitated.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An ultrasound diagnostic apparatus, comprising:
a cap whose outer periphery contacts a cavity wall directly or via a medium outside the cap; and
an ultrasound transducer including an electromechanical transducer element and an acoustic lens provided on the electromechanical transducer element, rotatable or swingable, capable of transmitting ultrasound with the electromechanical transducer element as a sound source and/or receiving echo from a subject area, and provided in the cap,
wherein
the lens focuses the ultrasound transmitted from the electromechanical transducer element and includes a shape in which a first focal length of a first lens surface on a section parallel with an ultrasound transducer rotational axis or a swing axis including a center of a sound beam axis of the ultrasound is shorter than a second focal length of a second lens surface on a section orthogonal to the ultrasound transducer rotational axis or the swing axis including the center of the sound beam axis.

2. The ultrasound diagnostic apparatus according to claim 1, comprising:
the cap whose outer periphery contacts the cavity wall directly or via the medium outside the cap;
the ultrasound transducer including the electromechanical transducer element and the lens in a concave shape provided on the electromechanical transducer element, rotatable or swingable around a center axis of the cap, capable of transmitting ultrasound with the electromechanical transducer element as the sound source and/or receiving echo from the subject area via the cap, and provided in the cap; and
an acoustic medium which is filled between an inner surface of the cap and the ultrasound transducer in the cap, and a sound velocity in the acoustic medium is lower than that in the medium outside the cap,
wherein
the acoustic lens focuses the ultrasound transmitted from the electromechanical transducer element, and includes a shape in which a first focal length of a first lens surface on a section parallel with the ultrasound transducer rotational axis or the swing axis including the center of the sound beam axis of the ultrasound is shorter than a second focal length of a second lens surface on a section orthogonal to the ultrasound transducer rotational axis or the swing axis including the center of the sound beam axis.

3. The ultrasound diagnostic apparatus according to claim 1,
wherein the ultrasound transducer further comprises a backing material provided on a side opposite from the lens of the electromechanical transducer element, and a housing which holds the electromechanical transducer element, the lens and the backing material, and configures a casing of the ultrasound transducer.

4. The ultrasound diagnostic apparatus according to claim 1,
wherein in the acoustic lens, the first lens surface and the second lens surface are formed into partial circular-arc shapes, and
a first radius of curvature of the first lens surface is formed to be smaller than a second radius of curvature of the second lens surface.

5. The ultrasound diagnostic apparatus according to claim 1,
wherein in the lens, the first lens surface is formed into a shape defined by an absolute value of a parabola or curve of third or higher order, and the second lens surface is formed into a partial circular-arc shape.

6. The ultrasound diagnostic apparatus according to claim 1,
wherein in the lens, the first lens surface and the second lens surface are formed into shapes defined by absolute values of a parabola or curve of third or higher order.

7. The ultrasound diagnostic apparatus according to claim 6,
wherein a first coefficient of the parabola defining the first lens surface is set to be larger than a second coefficient of the parabola defining the second lens surface.

8. The ultrasound diagnostic apparatus according to claim 1,
wherein in the lens, the first lens surface is formed into a shape defined by an absolute value of a parabola or curve of third or higher order, and the second lens surface is formed into a plane shape.

9. The ultrasound diagnostic apparatus according to claim 1,
the first lens surface and the second lens surface are formed into partial circular-arc shapes,
wherein
the first lens surface is formed into a shape with multiple steps from a plane having two radiuses of curvature including a third radius of curvature and a fourth radius of curvature differing from the third radius of curvature, and
the second lens surface is formed into a shape with multiple steps from a plane having two radiuses of curvature including a fifth radius of curvature larger than the third radius of curvature, and a sixth radius of curvature differing from the fifth radius of curvature and larger than the fourth radius of curvature.

10. The ultrasound diagnostic apparatus according to claim 1)
wherein the curved surface of the lens is formed by adding a second curve formula defining the second lens surface to a first curve formula defining the first lens surface.

11. The ultrasound diagnostic apparatus according to claim 1,
wherein the cap is formed into a cylindrical shape, and a diameter of the cap is 20 mm or less.

12. The ultrasound diagnostic apparatus according to claim 1, wherein the sound velocity of the medium outside the cap is 1500 m/sec or higher.

13. The ultrasound diagnostic apparatus according to claim 1,
wherein the medium outside the cap is water, or biologic fluid in the cavity.

14. The ultrasound diagnostic apparatus according to claim 1,
wherein a curvature of a cap on a section parallel with the ultrasound transducer rotational axis is different from a curvature of the cap on a section perpendicular to the ultrasound transducer rotational axis.

15. The ultrasound diagnostic apparatus according to claim l,
wherein the cap is formed into a spherical shape.

16. An endoscope, comprising.
a cap which is provided on a distal end of an insertion tube, and has an outer periphery contacting a cavity wall directly or via a medium outside the cap; and
an ultrasound transducer comprising an electromechanical transducer element, and a lens provided on the electromechanical transducer element, rotatable or swingable, capable of transmitting ultrasound with the electromechanical transducer element as a sound source and/or receiving echo from the subject area, and provided in the cap,
wherein
the lens focuses the ultrasound transmitted from the electromechanical transducer element, and includes a shape in which a first focal length of a first lens surface on a section parallel with an ultrasound transducer rotational axis or a swing axis including a center of a sound beam axis of the ultrasound is shorter than a second focal length of a second lens surface on a section orthogonal to the ultrasound transducer rotational axis or the swing axis including the center of the sound beam axis.

17. The endoscope according to claim 16, comprising:
the cap which is provided on the distal end of the insertion tube, and has the outer periphery contacting a cavity wall directly or via a medium outside the cap;
the ultrasound transducer comprising the electromechanical transducer element, and the lens in a concave shape provided in the electromechanical transducer element, rotatable or swingable around a center axis of the cap, capable of transmitting ultrasound with the electromechanical transducer element as a sound source and/or receiving echo from the subject area, and provided in the cap; and
an acoustic medium which is filled between an inner surface of the cap and the ultrasound transducer, has lower sound velocity than that in the medium outside the cap,
wherein
the lens focuses the ultrasound transmitted from the electromechanical transducer element, and includes a shape in which a first focal length of a first lens surface on a section parallel with the ultrasound transducer rotational axis or the swing axis including a center of a sound beam axis of the ultrasound is shorter than a second focal length of a second lens surface on a section orthogonal to the ultrasound transducer rotational axis or the swing axis including the center of the sound beam axis.

18. The endoscope according to claim 16,
wherein the ultrasound transducer further comprises a backing material provided on a side opposite from the lens of the electromechanical transducer element, and a housing which holds the electromechanical transducer element, the lens and the backing material, and configures a casing of the ultrasound transducer.

19. The endoscope according to claim 16,
wherein sound velocity of the medium outside the cap is 1500 m/sec or higher.

20. The endoscope according to claim 16,
wherein the medium outside the cap is water, or biologic fluid in the cavity.

21. An ultrasound transducer, comprising,
an ultrasound transducer unit comprising an electromechanical transducer element, a lens provided on the electromechanical transducer element, rotatable or swingable, and capable of transmitting ultrasound with the electromechanical transducer element as a sound source and/or receiving echo from a subject area, and provided in the cap,
wherein
the lens focuses the ultrasound transmitted from the electromechanical transducer element, and includes a shape in which a first focal length of a first lens surface on a section parallel with a ultrasound transducer rotational axis or a swing axis including a center of a sound beam axis of the ultrasound is shorter than a second focal length of a second lens surface on a section orthogonal to the ultrasound transducer rotational axis or the swing axis including the center of the sound beam axis.

22. The ultrasound transducer according to claim 21, comprising, the ultrasound transducer unit comprising the electromechanical transducer element, and the lens in a concave shape provided in the electromechanical transducer element, rotatable or swingable around a center axis of the cap, and capable of transmitting ultrasound with the electromechanical transducer element as a sound source and/or receiving echo from a subject area, and provided in the cap,
wherein
the lens focuses the ultrasound transmitted from the electromechanical transducer element, and includes a shape in which a first focal length of a first lens surface on a section parallel with the ultrasound transducer rotational axis or the swing axis including a center of a sound beam axis of the ultrasound is shorter than a second focal length of a second lens surface on a section orthogonal to the ultrasound transducer rotational axis or the swing axis including the center of the sound beam axis.

23. The ultrasound transducer according to claim 21,
wherein the ultrasound transducer unit further comprises a backing material provided on a side opposite from the lens of the electromechanical transducer element, and a housing which holds the electromechanical transducer element, the lens and the backing material, and configures a casing of the ultrasound transducer unit.
